(19) United States
(12) Patent Application Publication
(10) Pub. No.: US 2023/0148251 A1
(43) Pub. Date: May 11, 2023

(54) COMPOUND NUTRIENT CAPABLE OF REPAIRING DAMAGED INTESTINES OF PIGELETS AND METHOD OF USING THE SAME

(71) Applicant: Sichuan Agricultural University, Chengdu (CN)

(72) Inventors: Ping Zheng, Chengdu (CN); Daiwen Chen, Chengdu (CN); Bing Yu, Chengdu (CN); Jun He, Chengdu (CN); Jie Yu, Chengdu (CN)

(21) Appl. No.: 17/529,302

(22) Filed: Nov. 18, 2021

(30) Foreign Application Priority Data
Nov. 5, 2021 (CN) 202111306388.4

(51) Int. Cl. A61K 38/48; A61K 38/47; A61K 38/46; A61K 35/742; A61K 31/198; A61K 47/26; A61K 47/12; A61K 36/064; A61K 33/14; A61K 9/00; A61P 1/00; A23K 20/105; A23K 20/142; A23K 20/189; A23K 10/18; A23K 20/22; A23K 20/20; A23K 20/163; A23K 50/30; A23K 50/60

(52) U.S. Cl. CPC A61K 38/48; A61K 38/47; A61K 38/465; A61K 35/742; A61K 31/198; A61K 47/26; A61K 47/12; A61K 36/064; A61K 33/14; A61K 9/0056; A61P 1/00; A23K 20/105; A23K 20/142; A23K 20/189; A23K 10/18; A23K 20/22; A23K 20/30; A23K 20/163; A23K 50/30; A23K 50/60

(57) ABSTRACT

Disclosed provides a compound nutrient capable of repairing damaged intestinal tract of piglets and application thereof. The compound nutrient comprises the following components in weight percentage: organic acid 30-50%, amino acid 20-30%, enzyme preparation 8-15%, Bacillus subtilis 5-15%, mineral element 4-6%, glucose 5-20%. The enzyme preparation includes amylase, lipase and protease, and the weight ratio of amylase, lipase and protease is 1-2:1:1-2. The compound nutrient can repair the intestinal health of piglets with diarrhea, reduce the rate of diarrhea, and improve the growth performance.

### CROSS REFERENCE OF RELATED APPLICATIONS

The present application claims the benefit of China Patent Application No. 202111306388.4, entitled “COMPOUND NUTRIENT CAPABLE OF REPAIRING DAMAGED INTESTINES OF PIGLETS AND METHOD OF USING THE SAME” filed on Nov. 8, 2021, in the China National Intellectual Property Administration, the entire content of which is incorporated herein in its entirety.

### TECHNICAL FIELD

The present disclosure relates to a compound nutrient capable of repairing damaged intestines of piglets and method of using the same.

### BACKGROUND

Weaning is a crucial stage in the growth and development of piglets, during which the impact on the intestinal health of piglets may last a lifetime. Due to the imperfect digestion, absorption, and immune function development of piglets at weaning, they are extremely vulnerable to environmental, dietary, and psychological stress, resulting in a series of problems, namely “weaning syndrome”, such as dysbacteriosis low feed intake, high diarrhea rate, damage to the intestinal mucosa, nutritional metabolism disorders, and intestinal tract. The diarrhea rate of weaned piglets can reach 50% in 3˜5 days after weaning, which damages piglet's intestinal development and function, which is specifically manifested by the decrease of intestinal villi height, the increase of crypt depth, the thinning of mucus layer, and the increase of intestinal epithelial barrier permeability, the decrease of digestion and absorption function, and the unbalanced microbial flora in the intestinal tract. In addition, during the weaning period, the immunity of these piglets with diarrhea will decrease, the secondary prevalence will increase, and the intestinal injury will be further aggravated. If the intestinal injury is not influenceively repaired and the diarrhea is relieved, the growth of the piglet will be severely stagnated and death.

The main causes of piglet diarrhea are impaired intestinal development, weakened digestion, and disorder of intestinal flora. In order to repair the intestinal damage of weaned piglets and reduce the diarrhea rate of weaned piglets, the current methods include feeding milk replacer, supplementary feeding trough feed before weaning, feeding liquid feed, adding antibiotics to the diet, adding zinc oxide, adding plant extracts, and so on. Milk replacer products have a significant influence on improving the growth performance of weaned piglets, but they are expensive and cannot be widely used in production. At the same time, the lack of diet stimulates the piglet intestines, which is not conducive to the piglet intestinal development. Supplementary feeding trough material can alleviate weaning stress, but it does not promote the repair of intestinal damage. Liquid feeding can alleviate the stimulation of solid diets to weaned pigs and make the pigs survive the weaning period. However, the purchase of liquid feeding equipment requires a large amount of money. At present, most pig farms in China do not install liquid feeding production lines. Therefore, to relieve weaning stress and repair intestinal damage, nutritional means must be used. For a long time, adding antibiotics has become the main method to reduce diarrhea in weaned piglets. However, with the continuous emergence of drug-resistant strains and the decline in the immunity of piglets, the dosage of antibiotics has gradually increased. Over the long term, the abuse of antibiotics, bacterial resistance, and drug residues will seriously affect food safety and the health of consumers. At present, most European countries have banned the use of antibiotics as feed additives, and the Ministry of Agriculture of China has also issued a policy of non-resistant breeding, which clearly stipulates that antibiotics are prohibited from being added to diets from Jul. 1, 2020. Zinc oxide can influenceively enhance the immune function of the body and promote the growth of piglets, but most of the zinc oxide will be excreted with feces, causing waste of zinc resources and environmental pollution. The Announcement No. 2625 of the Ministry of Agriculture of the People's Republic of China has been officially implemented on Jul. 1, 2018, beginning to reduce zinc in an all-round way. Plant extracts are considered to be green and safe feed additives in recent years, which can influenceively alleviate the weaning stress of piglets, but their components are complex and their potential impact on piglets is still unknown. Therefore, the prior art cannot meet the nutritional needs of piglets with intestinal damage after weaning. It is imperative to develop a safe, pollution-free, and residue-free feed additive that relieves piglet diarrhea, repairs intestinal injury, and promotes piglet growth.

### SUMMARY OF THIS DISCLOSURE

The object of the present disclosure is to provide a compound nutrient capable of repairing damaged intestines of piglets and method of using the same. The compound nutrient is capable of recovering the intestinal health of piglets with diarrhea, reducing the rate of diarrhea, and improving the growth performance.

The compound nutrient capable of repairing damaged intestines of piglets and method of using the same comprises the following, by weight percentage:

| | |
|---|---|
| organic acid | 30%-50% |
| amino acid | 20%-30% |
| enzyme preparation | 8%-15% |
| bacillus subtilis | 5%-15% |
| mineral elements | 4%-6% |
| glucose | 5%-20% |

where, the enzyme preparation comprises amylase, lipase and protease, and the weight ratio of the amylase, lipase and protease in the enzyme preparation is 1-2:1:1-2.

Preferably, the compound nutrient comprises the following, by weight percentage:

| | |
|---|---|
| organic acid | 40% |
| amino acid | 24% |
| enzyme preparation | 10% |
| bacillus subtilis | 10% |
| mineral elements | 6% |
| glucose | 10% |

Preferably, the weight ratio of the amylase, lipase and protease in the enzyme preparation is 1:1:2.

Preferably, the amino acid comprises glutamine and arginine, and the weight ratio of glutamine and arginine is 2-5:1.

Preferably, the weight ratio of glutamine and arginine is 5:1.

Preferably, the organic acid comprises lactic acid, citric acid and fumaric acid, and the weight ratio of the lactic acid, the citric acid and the fumaric acid is 1-6:1-2:1.

Preferably, the weight ratio of the lactic acid, the citric acid and the fumaric acid is 6:2:1.

Preferably, the mineral element comprises potassium chloride and yeast selenium, and the weight ratio of the potassium chloride to yeast selenium is 50-100:1.

Preferably, the weight ratio of the potassium chloride to yeast selenium is 50:1.

The present disclosure further provides a fodder capable of repairing damaged intestines of piglets. The fodder comprises the compound nutrient as described above.

Preferably, the weight percentage of the compound nutrient in the fodder is 1%.

The present disclosure further provides the application of the above-mentioned compound nutrients in the preparation of feed additives or fodder capable of repairing damaged intestines of piglets to improve growth performance of piglets or reducing the incidence of piglet diarrhea.

The present disclosure has the following advantages.

According to the characteristics of nutrients and the principle of functional complementarity and superposition, the present disclosure obtains a green, non-resistant compound nutrient through inventive and novel research and experiments. The compound nutrient can repair the damaged intestines of piglets and prevent piglet diarrhea, thereby improving the growth performance of piglets and reducing the incidence of piglet diarrhea.

### BRIEF DESCRIPTION OF THE DRAWINGS

FIG. 1 show the influence of enzyme preparation on the level of feed acid-soluble protein.

FIG. 2 shows the influence of enzyme preparation on the content of feed lactic acid.

### DETAILED DESCRIPTION OF THE DISCLOSURE

In order to better illustrate the objectives, technical solutions and advantages of the present disclosure, the present disclosure will be further described below in conjunction with specific embodiments.

### Embodiment 1

The present disclosure first investigated the influence of enzyme preparation, organic acid and amino acid on the growth performance, diarrhea rate and intestinal health of piglets with diarrhea.

1. The Influence of Enzyme Preparation on Quality of Fermented Feed.

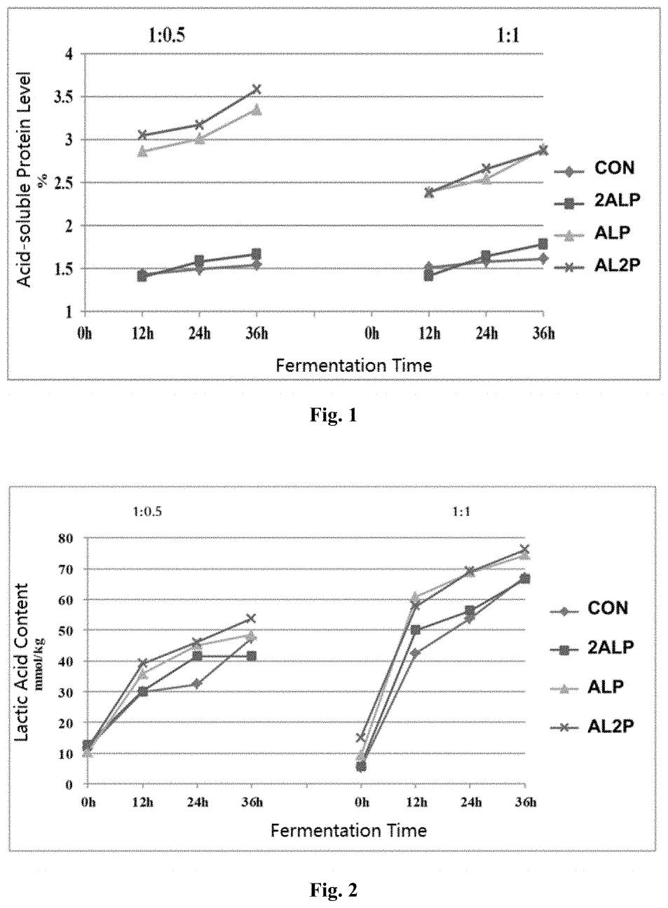

This experiment evaluates the influence of adding enzyme preparations on the quality of fermented feed. The experiment was designed with 4 treatments: control group (CON) (without enzyme preparation), 2ALP group (with 0.1% compound enzyme added, and amylase: lipase: protease=2:1:1), ALP group (with 0.1% compound enzyme added, and amylase: lipase: protease=1:1:1), AL2P group (add 0.1% compound enzyme, and amylase: lipase: protease=1:1:2). Under the fermentation process parameters screened in the laboratory, the influence of enzyme preparations on the acid-soluble protein content of fermented diets were investigated under the conditions of feed-water ratios of 1:0.5 and 1:1. Fermented diets: 30.28% expanded corn, 35.69% corn, 11.89% soybean meal, and 11.89% Expanded soybeans, 2.38% sucrose, 2.14% soybean oil. The results showed that amylase:lipase:protease=1:1:2 significantly increased the content of feed acid-soluble protein and lactic acid (FIG. 1 and FIG. 2).

The AL2P group feed was fed to piglets. Compared with the control group without enzyme preparation, the results are shown in Table 1. The daily gain of weaned piglets increased by 15% and the diarrhea rate was reduced by 52%.

TABLE 1
Results of daily gain and diarrhea rate for weaned piglets

| Items | Con | AL2P | P-value |
|---|---|---|---|
| Initial BW (kg) | 7.20 | 7.20 | 1.00 |
| 21 d BW (kg) | 8.93 | 9.24 | 0.42 |
| ADG (g) | 123 | 145 | 0.08 |
| Diarrhea rate % | 12.50 | 5.95 | 0.20 |

2. The Influence of Different Organic Acid Combinations on Fecal Microbes and Diarrhea Rate of Piglets.

The 21-day-old weaned piglets were randomly divided into five treatments, with 10 replicates in each group and 1 piglet in each replicate. They were fed the control diet, treatment 1 (addition of 0.4% organic acid, i.e. lactic acid+citric acid diet (1:1)), treatment 2 (addition of 0.4% organic acid, i.e. lactic acid+citric acid+fumaric acid diet (1:1:1)), treatment 3 (addition of 0.4% organic acid, i.e. lactic acid+citric acid+rich Maric acid (3:2:1)), treatment 4 (addition of 0.4% organic acid, i.e. lactic acid+citric acid+fumaric acid (6:2:1)). The test period was 21 days, and the composition of the microbes in piglet feces was investigated. The results showed that compared with the control group (CON) (Table 2), the combination of treatment 4 lactic acid+citric acid+fumaric acid (6:2:1) significantly reduced the number of Escherichia coli in piglet manure, significantly increased the number of lactobacilli, and reduced the diarrhea rate of piglets.

TABLE 2
Influence of different combinations of organic acids on
the fecal microflora and diarrhea rate of weaned piglets

| Items | Con | T1 | T2 | T3 | T4 | SEM | P-value |
|---|---|---|---|---|---|---|---|
| Total bacteria | 9.71 | 9.65 | 9.72 | 9.73 | 9.84 | 0.16 | 0.83 |
| Lactobacillus | 3.39^{b} | 3.51^{ab} | 3.52^{ab} | 3.66^{ab} | 3.78^{a} | 0.16 | 0.04 |
| E. coli | 6.97^{a} | 6.75^{ab} | 6.71^{ab} | 6.16^{b} | 5.85^{b} | 0.17 | 0.03 |
| Diarrhea rate % | 10.05^{a} | 8.54^{a} | 7.66^{ab} | 4.35^{b} | 4.40^{b} | 0.85 | 0.09 |

3. The Influence of Supplementing Arginine on the Growth Performance of Weak Piglets.

Three-day-old suckling piglets with low birth weight were used as experimental animals. They were randomly divided into two treatments, fed with 0.04% and without arginine feed. Each treatment had 8 replicates, and each replicate had 1 piglet. The test period was 21 days to investigate the growth performance of piglets. The results show (Table 3) that arginine supplementation can significantly improve the daily gain and feed intake of weak piglets.

TABLE 3
Influence of supplementation of arginine in diet on growth
performance of low-birth-weight suckling piglets

| Items | Con | Arginine Group | P-value |
|---|---|---|---|
| Initial BW (kg) | 1.17 ± 0.03 | 1.16 ± 0.03 | 1.000 |
| 21 d BW (kg) | 5.50 ± 0.09 | 6.24 ± 0.15 | 0.02 |
| ADG (g) | 207 ± 3 | 242 ± 6 | 0.03 |
| ADFI (g) | 144 ± 3 | 174 ± 5 | 0.04 |
| F/G | 0.70 ± 0.02 | 0.72 ± 0.01 | 0.55 |

4. The Influence of Arginine Supplementation on the Anti-Oxidation Ability of Weak Piglets.

Three-day-old suckling piglets with low birth weight were used as experimental animals. They were randomly divided into two treatments, fed with 0.04% and without arginine feed. Each treatment had 8 replicates, and each replicate had 1 piglet. The test period was 21 days. After the test, the piglet serum was collected to investigate the antioxidant capacity of the piglet serum. The results show (Table 4) that arginine supplementation can significantly improve the antioxidant capacity of weak piglets.

TABLE 4
Influence of diet supplementation with arginine
on serum antioxidant enzyme activity and MDA
content in low-birth-weight suckling piglets

| Items | Con | Arginine Group | P-value |
|---|---|---|---|
| MAD (nmol/mL) | 1.32 ± 0.12 | 0.97 ± 0.14 | 0.049 |
| CAT (U/mL) | 11.83 ± 1.03 | 16.28 ± 0.91 | 0.027 |
| SOD (U/mL) | 64.49 ± 2.47 | 70.66 ± 2.78 | 0.038 |
| GPx (U/mL) | 163.62 ± 15.66 | 194.33 ± 17.03 | 0.129 |

5. The Influence of Supplementation of Arginine and Glutamine on the Growth Performance of Weaned Piglets.

The 25-day-old weaned piglets were used as experimental animals. They were randomly divided into three treatments. They were fed with no addition (control group), addition of 0.10% arginine and 0.20% glutamine (A2G), and addition of 0.04% arginine and 0.20% glutamine feed (A5G), 10 replicates in each treatment, 2 piglets in each replicate, and the test period is 7 days to investigate the growth performance and diarrhea rate of the piglets. The results show (Table 5 and Table 6) that supplementation of arginine and glutamine can significantly improve the daily gain of weaned piglets, increase the height of jejunum villi, and promote jejunum development.

TABLE 5
Influence of supplementation of arginine and glutamine
in diet on growth performance of weaned piglets

| Items | Con | A2G | A5G | SEM | P-value |
|---|---|---|---|---|---|
| Initial BW (kg) | 7.25 | 7.25 | 7.25 | 0.03 | 0.99 |
| 7 d BW (kg) | 8.26 | 8.49 | 8.55 | 0.05 | 0.38 |
| ADFI (g) | 275 | 298 | 296 | 5.79 | 0.22 |
| ADG (g) | 144 | 177 | 186 | 5.17 | 0.08 |
| Diarrhea rate % | 8.67 | 6.25 | 6.31 | 1.26 | 0.56 |

TABLE 6
Influence of supplementation of arginine and glutamine
on jejunum morphology of weaned piglets

| Items | Con | A2G | A5G | SEM | P-value |
|---|---|---|---|---|---|
| Villus height (μm) | 335^{b} | 376^{a} | 382^{a} | 15 | 0.03 |
| Crypt depth (μm) | 175 | 163 | 161 | 5 | 0.91 |

### Embodiment 2

The present disclosure studies and screens the repairing influence of compound nutrients composed of organic acids, amino acids, enzyme preparations, probiotics, mineral elements, etc. on the intestinal injury of diarrhea piglets on the basis of preliminary research. The present disclosure investigates the influence of compound nutrients on the growth performance, diarrhea rate and intestinal health of piglets with diarrhea through piglet feeding experiments combined with biochemical analysis methods.

The present disclosure has carried out a systematic study. The present disclosure takes 21-day-old weaned piglets as experimental animals, and feeds piglets with 0 and 1% of the compound nutrient combination additive of the present disclosure. The growth performance, diarrhea rate, serum biochemical, intestinal Tract permeability, jejunum shape, etc.

The compound nutrient of the present disclosure comprises the following by weight percentage:

| | |
|---|---|
| organic acid | 10% |
| amino acid | 24% |
| enzyme preparation | 10% |
| bacillus subtilis | 10% |
| mineral elements | 6% |
| glucose | 10% |

where, organic acid comprises lactic acid, citric acid and fumaric acid, and the weight ratio of lactic acid, citric acid and fumaric acid is 6:2:1; the amino acid comprises glutamine and arginine, and the weight ratio of glutamine and arginine is 5:1; the enzyme preparation comprises amylase, lipase and protease, and the weight ratio of amylase, lipase and protease is 1:1:2; and the mineral elements include potassium chloride and yeast selenium, and the weight ratio of potassium chloride to yeast selenium is 50:1.

The ability of the compound nutrient of the present disclosure to repair the damaged intestines of the piglets was verified on the diarrhea piglets. The 21-day-old weaned piglets were fed with 4% spoiled fish meal and then the piglets that developed diarrhea were used as experimental animals. They were randomly divided into two treatments and fed separately. Feed the feed supplemented with 0 and 1% of the additive of the present disclosure. Each group has 10 repetitions, and each repetition is 1 piglet. The test period is 28 days to investigate the growth performance, diarrhea rate, serum biochemistry, intestinal permeability, and jejunal villi morphology of the piglets. The test diet was prepared according to the NRC (2012) feeding requirements standard, and the nutrient level in the test diet could meet or exceed the nutritional needs of the piglets.

TABLE 7
Influence of the present disclosure on the production performance
and diarrhea rate of weaned diarrhea piglets

| Items | Con | Test Group | SEM | P-value |
|---|---|---|---|---|
| Initial BW (kg) | 7.23 | 7.23 | 0.30 | 1.00 |
| End BW (kg) | 17.06 | 19.27 | 0.75 | 0.05 |
| ADG ( ) | 351 | 430 | 28 | 0.04 |
| ADFI | 565 | 643 | 32 | 0.03 |
| Feed-to-Weight | 1.61 | 1.50 | 0.04 | 0.03 |
| Diarrhea rate % | 0.62 | 0.28 | 0.08 | <0.01 |

The results showed (Table 7) that the test group significantly increased the daily weight gain and average daily feed intake of piglets, and now reduced the feed-to-weight ratio, which significantly reduced the diarrhea rate of piglets.

TABLE 8
Influence of the present disclosure on serum
biochemistry of piglets with weaning diarrhea

| Items | Con | Test Group | SEM | P-value |
|---|---|---|---|---|
| Cr/(μmol/L) | 106 | 94 | 6 | 0.07 |
| BUN/(mmol/L) | 10.2 | 8.1 | 0.8 | 0.02 |
| AST/(U/gprot) | 13.5 | 11.8 | 2.1 | 0.75 |
| ALT (U/gprot) | 10.5 | 11.6 | 1.3 | 0.86 |
| MDA (mmol/mL) | 4.6 | 1.9 | 0.5 | 0.01 |

The results showed (Table 8) that the test group significantly reduced the levels of serum urea nitrogen and malondialdehyde in piglets.

TABLE 9
Influence of the present disclosure on the intestinal
permeability of weaning diarrhea (%)

| Items | Con | Test Group | SEM | P-value |
|---|---|---|---|---|
| D-LA/(μg/L) | 226 | 199 | 7 | 0.02 |
| DAO/(pg/mL) | 47 | 44 | 0.5 | 0.03 |

The results showed (Table 9) that the test group significantly reduced the level of serum D-lactic acid and the activity of diamine oxidase in piglets.

TABLE 10
Influence of the present disclosure on the morphology
of jejunum villi of weaned diarrhea piglets (%)

| Items | Con | Test Group | SEM | P-value |
|---|---|---|---|---|
| Villus height | 220 | 254 | 7 | <0.01 |
| Crypt depth/(μm) | 109 | 104 | 5 | 0.92 |
| V/C | 2.0 | 2.2 | 0.1 | 0.58 |

The results showed (Table 10) that the experimental group significantly increased the height of intestinal villi of piglets with diarrhea.

Finally, it should be noted that the above embodiments are only used to illustrate the technical solutions of the present disclosure and not to limit the protection scope of the present disclosure. Although the present disclosure has been described in detail with reference to the preferred embodiments, those of ordinary skill in the art should understand that the technical solution of the present disclosure can be modified or equivalently replaced without departing from the essence and scope of the technical solution of the present disclosure.

What is claimed is:

1. A compound nutrient capable of repairing damaged intestines of piglets, comprising the following, by weight percentage:

| | |
|---|---|
| organic acid | 30%-50% |
| amino acid | 20%-30% |
| enzyme preparation | 8%-15% |
| bacillus subtilis | 5%-15% |
| mineral elements | 4%-6% |
| glucose | 5%-20% |

wherein, the enzyme preparation comprises amylase, lipase and protease, and the weight ratio of the amylase, lipase and protease in the enzyme preparation is 1-2:1:1-2.

2. The compound nutrient according to claim 1, wherein the compound nutrient comprises the following, by weight percentage:

| | |
|---|---|
| organic acid | 40% |
| amino acid | 24% |
| enzyme preparation | 10% |
| bacillus subtilis | 10% |
| mineral elements | 6% |
| glucose | 10% |

3. The compound nutrient according to claim 1, wherein the weight ratio of the amylase, lipase and protease in the enzyme preparation is 1:1:2.

4. The compound nutrient according to claim 1, wherein the amino acid comprises glutamine and arginine, and the weight ratio of glutamine and arginine is 2-5:1.

5. The compound nutrient according to claim 1, wherein the weight ratio of glutamine and arginine is 5:1.

6. The compound nutrient according to claim 1, wherein the organic acid comprises lactic acid, citric acid and fumaric acid, and the weight ratio of the lactic acid, the citric acid and the fumaric acid is 1-6:1-2:1.

7. The compound nutrient according to claim 1, wherein the weight ratio of the lactic acid, the citric acid and the fumaric acid is 6:2:1.

8. The compound nutrient according to claim 1, wherein the mineral element comprises potassium chloride and yeast selenium, and the weight ratio of the potassium chloride to yeast selenium is 50-100:1.

9. The compound nutrient according to claim 1, wherein the weight ratio of the potassium chloride to yeast selenium is 50:1.

10. A fodder capable of repairing damaged intestines of piglets, comprising the compound nutrient of claim 1.

11. The fodder according to claim 1, wherein the weight percentage of the compound nutrient in the fodder is 1%.

12. A feed additive or feed for repairing damaged intestines of piglets, improving growth performance of piglets or reducing the incidence of piglet diarrhea, comprising the compound nutrient of claim 1.
